SEQUENCE LISTING (4 sequences)

SEQ ID NO: 1 - protein, 590 aa, Homo sapiens
MKVGVLWLISFFTFTDGHGGFLGKNDGIKTKKELIVNKKKHLGPVEEYQLLLQVTYRDSKEKRDLRNFLKLLKPPLLWSHGLIRIIRAKATTDCNSLNGVLQCTCEDSYTWFPPSCLDPQNCYLHTAGALPSCECHLNNLSQSVNFCERTKIWGTFKINERFTNDLLNSSSAIYSKYANGIEIQLKKAYERIQGFESVQVTQFRNGSIVAGYEVVGSSSASELLSAIEHVAEKAKTALHKLFPLEDGSFRVFGKAQCNDIVFGFGSKDDEYTLPCSSGYRGNITAKCESSGWQVIRETCVLSLLEELNKNFSMIVGNATEAAVSSFVQNLSVIIRQNPSTTVGNLASVVSILSNISSLSLASHFRVSNSTMEDVISIADNILNSASVTNWTVLLREEKYASSRLLETLENISTLVPPTALPLNFSRKFIDWKGIPVNKSQLKRGYSYQIKMCPQNTSIPIRGRVLIGSDQFQRSLPETIISMASLTLGNILPVSKNGNAQVNGPVISTVIQNYSINEVFLFFSKIESNLSQPHCVFWDFSHLQWNDAGCHLVNETQDIVTCQCTHLTSFSILMSPFVPSTIFPVVKWITY

SEQ ID NO: 2 - protein, 910 aa, Homo sapiens
MKVGVLWLISFFTFTDGHGGFLGKNDGIKTKKELIVNKKKHLGPVEEYQLLLQVTYRDSKEKRDLRNFLKLLKPPLLWSHGLIRIIRAKATTDCNSLNGVLQCTCEDSYTWFPPSCLDPQNCYLHTAGALPSCECHLNNLSQSVNFCERTKIWGTFKINERFTNDLLNSSSAIYSKYANGIEIQLKKAYERIQGFESVQVTQFRNGSIVAGYEVVGSSSASELLSAIEHVAEKAKTALHKLFPLEDGSFRVFGKAQCNDIVFGFGSKDDEYTLPCSSGYRGNITAKCESSGWQVIRETCVLSLLEELNKNFSMIVGNATEAAVSSFVQNLSVIIRQNPSTTVGNLASVVSILSNISSLSLASHFRVSNSTMEDVISIADNILNSASVTNWTVLLREEKYASSRLLETLENISTLVPPTALPLNFSRKFIDWKGIPVNKSQLKRGYSYQIKMCPQNTSIPIRGRVLIGSDQFQRSLPETIISMASLTLGNILPVSKNGNAQVNGPVISTVIQNYSINEVFLFFSKIESNLSQPHCVFWDFSHLQWNDAGCHLVNETQDIVTCQCTHLTSFSILMSPFVPSTIFPVVKWITYVGLGISIGSLILCLIIEALFWKQIKKSQTSHTRRICMVNIALSLLIADVWFIVGATVDTTVNPSGVCTAAVFFTHFFYLSLFFWMLMLGILLAYRIILVFHHMAQHLMMAVGFCLGYGCPLIISVITIAVTQPSNTYKRKDVCWLNWSNGSKPLLAFVVPALAIVAVNFVVVLLVLTKLWRPTVGERLSRDDKATIIRVGKSLLILTPLLGLTWGFGIGTIVDSQNLAWHVIFALLNAFQGFFILCFGILLDSKLRQLLFNKLSALSSWKQTEKQNSSDLSAKPKFSKPFNPLQNKGHYAFSHTGDSSDNIMLTQFVSNE

SEQ ID NO: 3 - protein, 19 aa, Homo sapiens
DLFIDKKVLKVAHVEHEET

SEQ ID NO: 4 - protein, 16 aa, Homo sapiens
LAYDLDVDDAPGNSQQ

(19) United States
(12) Patent Application Publication
(10) Pub. No.: US 2013/0224209 A1
(43) Pub. Date: Aug. 29, 2013

(54) METHODS FOR DIAGNOSING AND TREATING PROSTATE AND LUNG CANCER

(71) Applicant: PICOBELLA, LLC, (US)

(72) Inventors: Bruce Wang, Mountain View, CA (US); Matthias Wabl, San Francisco, CA (US)

(73) Assignee: PICOBELLA, LLC, Burlingame, CA (US)

(21) Appl. No.: 13/746,180

(22) Filed: Jan. 21, 2013

Related U.S. Application Data
(63) Continuation of application No. 13/421,518, filed on Mar. 15, 2012, now abandoned.
Application No. 13/421,518 is a continuation of application No. 13/218,796, filed on Aug. 26, 2011, now abandoned.
Application No. 13/218,796 is a continuation of application No. 12/599,263, now abandoned.
(60) Provisional application No. 60/916,719, filed on May 8, 2007.

(51) Int. Cl. C12Q 1/68; G01N 33/68

(52) U.S. Cl. CPC C12Q 1/6886; G01N 33/6893
USPC 424/139.1; 435/7.23; 435/287.2; 435/6.12

(57) ABSTRACT

Methods for detecting and treating prostate and lung cancer are disclosed In practicing the method, a subject sample is assayed for GPR110 protein or its RNA transcript, and the GPR110 or transcript level observed is used in determining whether the subject has an elevated GPR110 level associated with prostate or lung cancer. Patients with such elevated levels may be treated, in accordance with the invention, with a variety of GPR110 related immunotherapy agents.

### FIELD OF THE INVENTION

The present invention relates to a gene and encoded protein related to prostate and lung cancer, and to methods and reagents for detecting and treating prostate and lung cancer.

### REFERENCES

The following references are cited below in support of the background of the invention or methods employed in practicing the invention.

1. Bjarnadottir T K, Geirardsdottir K, Ingemansson M, Mirza M A, Fredriksson R, Schioth H B. Identification of novel splice variants of Adhesion G protein-coupled receptors. Gene. 2007 January 31; 387(1-2):38-48. Epub 2006 Aug. 30.

2. Bjarnadottir T K, Fredriksson R, Hoglund P J, Gloriam D E, Lagerstrom M C, Schioth H B. The human and mouse repertoire of the adhesion family of G-protein-coupled receptors. Genomics. 2004 July; 84(1):23-33.

3. Fredriksson R, Lagerstrom M C, Hoglund P J, Schioth H B. Novel human G protein-coupled receptors with long N-terminals containing GPS domains and Ser/Thr-rich regions. FEBS Lett. 2002 Nov. 20; 531(3):407-14.

4. Nusse, R., van Ooyen, A., Cox, D., Fung, Y. K. & Varmus, H. Mode of proviral activation of a putative mammary oncogene (int-1) on mouse chromosome 15. Nature 307, 131-6 (1984).

5. Nusse, R. & Varmus, H. E. Many tumors induced by the mouse mammary tumor virus contain a provirus integrated in the same region of the host genome. Cell 31, 99-109 (1982).

6. Sorensen, A. B., Duch, M., Amtoft, H. W., Jorgensen, P. & Pedersen, F. S. Sequence tags of provirus integration sites in DNAs of tumors induced by the murine retrovirus SL3-3. J Virol 70, 4063-70 (1996).

7. Lund, A. H. et al. Genome-wide retroviral insertional tagging of genes involved in cancer in Cdkn2a-deficient mice. Nat Genet 32, 160-5 (2002).

8. Mikkers, H. et al. High-throughput retroviral tagging to identify components of specific signaling pathways in cancer. Nat Genet 32, 153-9 (2002).

9. Collier, L. S., Carlson, C. M., Ravimohan, S., Dupuy, A. J. & Largaespada, D. A. Cancer gene discovery in solid tumours using transposon-based somatic mutagenesis in the mouse. Nature 436, 272-6 (2005).

10. Dupuy, A. J., Akagi, K., Largaespada, D. A., Copeland, N. G. & Jenkins, N. A. Mammalian mutagenesis using a highly mobile somatic Sleeping Beauty transposon system. Nature 436, 221-6 (2005).

11. Wang, et al., Nucleic Acids Research, (England) 2005, Vol. 33, p. 21.

12. Oh da Y, Kim K, Kwon H B, Seong J Y. Cellular and molecular biology of orphan G protein-coupled receptors. Int Rev Cytol. 2006; 252:163-218.

13. Lundstrom K. Latest development in drug discovery on G protein-coupled receptors. Curr Protein Pept Sci. 2006 October; 7(5):465-70.

14. Jacoby E, Bouhelal R, Gerspacher M, Seuwen K. The 7 TM G-protein-coupled receptor target family. ChemMedChem. 2006 August; 1(8):761-82.

### BACKGROUND OF THE INVENTION

Prostate cancer is the most common malignant cancer in North American men. It is estimated that approximately 200,000 new cases and 31,500 prostate cancer-related deaths will occur in the United States annually. Prostate cancer is now the second leading cause of cancer death in men, exceeded only by lung cancer. It accounts for 29% of all male cancers and 11% of male cancer-related deaths.

Currently, the FDA has approved serum PSA (prostate-specific antigen) for use as a prostate cancer screening laboratory test. Like many serum tumor markers, PSA is produced by both normal and cancerous glands. In men with prostate cancer, the serum levels can be elevated with both localized and advanced or disseminated disease. PSA levels are generally proportional to the volume of the cancer. Because there is a significant overlap between PSA levels found in cancer and benign prostatic hyperplasia, it is important to obtain sequential levels in low or borderline elevated values.

The introduction of free PSA (fPSA) testing has introduced a greater level of specificity in identifying early prostate cancer. In 1998, the FDA approved fPSA testing as a diagnostic aid for men with total PSA values between 4.0-10.0 ng/mL. This has often been the diagnostic gray zone for total PSA testing and fPSA may aid in the stratification. In general, at any free PSA level, the more enlarged the prostate, the more likely the prostate may be cancerous. However, these tests remain qualitative at best, and more reliable types of detection, and means for staging the cancer treatment, are needed.

Prostate cancer, like other forms of cancer, is caused by genetic aberrations, i.e., mutations. In mutant cells the normal balance between the factors that promote and restrain growth is disrupted, and as a result, these mutant cells proliferate continuously-the hallmark of tumor cells. Mutations can arise spontaneously or by external factors such as chemical mutagens, radiation, or viral integration, which inserts extra-genomic DNA that may or may not contain an oncogene. A cellular gene can be modified by point mutation, insertion and frame shift (including truncation), (functional) deletion (including silencing), or translocation, which sometimes can result in gene fusion. In this way proto-oncogenes can become oncogenes, which promote proliferation, and tumor suppressor genes can become inactivated, also inducing tumor growth. Any combination of the above-mentioned changes in DNA can contribute to tumor formation. The consequences of these changes may or may not be held in check by the immune system (immune surveillance).

Heretofore, there has been no demonstrated link between changes in GPR110 levels and prostate or lung cancer. Such a link could have a number of important diagnostic and therapeutic applications. In accordance with the present invention, it has now been discovered that (i) GPR110 levels increase significantly in prostate and lung cancer cells, and (ii) this increase can be measured in blood or urine-fluid sample of patients.

### SUMMARY OF THE INVENTION

The invention includes, in one aspect, a method for screening for lung or prostate cancer in a human subject. The method includes the steps of:(a) assaying the level of human GPR110 or its RNA transcript in a subject sample, (b) determining if the assayed level of human GPR110 or its RNA transcript is at least threefold greater than the level of GPR110 or its transcript, respectively, in a normal human subject, as determined from a plurality of normal human samples. Optionally, the method may include screening for the presence of lung or prostate cancer by means of an independent test for lung or prostate cancer, respectively, in the subject, if the assayed level is at least threefold greater than normal level, where the independent test may be carried out prior to, contemporaneous with, or following steps (a) and (b).

Where the subject sample is a lung or prostate histological tissue sample, step (a) may include contacting the sample with an anti-GPR110 antibody specific against a GPR110 epitope, under conditions effective to bind the antibody to cells having the GPR110 epitope, and detecting the level of antibody associated with said sample, and step (b) may include determining if the detected level of antibody associated with the subject lung or prostate tissue sample is at least threefold greater than that of anti-GPR110 antibody associated with human lung or prostate tissue samples, respectively, obtained from normal individuals. The antibody may be specific against a GPR110 epitope represented by amino acid residues within SEQ ID NO:1. The antibody may be a radiolabeled GPR110 antibody, and step (a) may include detecting the level of localized radiolabel scintigraphically in said tissue.

Where the subject sample is a subject blood or serum sample, step (a) may include contacting the sample with an anti-GPR110 antibody specific against a GPR110 epitope, under conditions effective to bind the antibody to the GPR110 epitope, separating antibody bound to the GPR110 epitope from unbound antibody, and detecting the level of the antibody bound to the GPR110 epitope, and step (b) may include determining if the detected level of antibody bound to the GPR110 epitope is at least threefold greater than that of anti-GPR110 antibody bound to the GPR110 epitope present in blood or serum samples obtained from normal individuals. Step (a) may include applying the blood or serum sample body fluid to a solid-phase immunoassay device, where the level of GPR110 in the sample is indicated qualitatively by a colorimetric or fluorometric indicator, and the determining step includes comparing the indicator with a known standard.

Where the subject sample is a lung or prostate tissue sample, step (a) may include processing the sample to extract RNA transcript therefrom and detecting the level of RNA transcript encoding for at least a fragment of GPR110 protein, and step (b) may include determining if the detected level of RNA transcript is at least threefold greater than the detected level of transcript encoding for at least a fragment of GPR110 protein in lung or prostate tissue samples obtained from normal individuals.

In another aspect, the invention includes an improvement in a method for detecting the presence of lung or prostate cancer, by detection of a depressed or elevated level of a biological marker that is diagnostic of the lung or prostate cancer. The improvement comprises the steps of: (a) assaying the level of human GPR110 or its transcript in a subject sample, and (b) determining if the assayed level of human GPR110 or its transcript is at least threefold greater than the level of GPR110 or its transcript, respectively, in a normal human subject, as determined from a plurality of normal human samples, as an additional indicator of the presence of lung or prostate cancer, respectively. Various preferred embodiments of the method noted above apply to this aspect of the invention as well.

For example, the improvement may be used in a method for detecting prostate cancer in a human male subject, by reacting a subject body-fluid sample with an antibody specific against at least one marker protein selected from one of total prostate specific antigen (PSA), free PSA, and glypican 3 protein (GPC3), and determining, as an indicator of prostate cancer, whether the subject has an increased level of at least one of said marker protein.

In still another aspect, the invention contemplates the use of a measured value of GPR110 and a measured value of at least one marker antigen selected from total prostate specific antigen (PSA), free PSA, and glypican 3 protein (GPC3) in a blood or serum sample from a human subject for screening the subject for the presence of prostate cancer.

Also disclosed is a diagnostic device for use in the screening for prostate or lung cancer in a human subject, or staging treatment of prostate or lung cancer in a subject, comprising (a) structure for receiving a body-fluid sample from the subject, (b) an antibody specific against a selected domain or epitope of GPR110, and associated with said structure and capable of reacting with body-fluid received in said structure, to produce, in combination with other reagents associated with the structure, a detectable reaction indicative of the presence of GPR110 sample protein containing that epitope or domain, and (c) a known-standard indicator against which the level of detectable reaction produced can be assessed as an increased level associated with prostate or lung cancer. The device may be applied more generally in screening for or staging other types of human cancer characterized by an increased level of GPR110.

The structure in the device may include a porous pad having the antibody embedded therein, for reaction with the fluid sample when the sample is applied to the pad, the detectable reaction may be indicated by a colorimetric or fluorimetric indicator, and the known standard indicator may include an indicia that represents a level of GPR110 containing the epitope or domain corresponding to that associated with prostate or lung cancer.

The device may include a spectrophotometric detector for generating a signal related to the level of GPR110 produced, a microprocessor for comparing the signal with a known-standard signal value associated with prostate or lung cancer, and a display for displaying an output of the microprocessor.

The anti-GPR110 binding protein in the device may be an antibody specific against an epitope contained within SEQ ID NO:1 or SEQ ID NO:2.

For use in the screening for prostate cancer in a human subject, element (b) in the device may further include an antibody that is (i) specific against at least one marker protein selected from one of total prostate specific antigen (PSA), free PSA, and glypican 3 protein (GPC3), (ii) associated with said structure and (iii) capable of reacting with body-fluid received in said structure, to produce, in combination with other reagents associated with the structure, a detectable reaction indicative of the level of the marker protein in the sample, and element (c) may further include a second known-standard indicator against which the level of detectable marker protein reaction produced can be assessed, in combination with the level of detectable GPR110, as an indicator of prostate cancer. The two standard indicators may be arranged, for example, in pairs of values, each pair representing a predetermined likelihood of prostate cancer in a subject.

Also disclosed is a method for treating prostate or lung cancer in a subject, by the steps of: (a) determining whether cancer tissue cells from the subject have an increased level of GPR110 protein or RNA transcript, when compared with a normal range of GPR110 protein or RNA transcript, respectively, in human cells of the same tissue, as an indicator of prostate or lung cancer, and (b) if the subject has such an increased GPR110 level, administering a therapeutically effective amount of a GPR110 antibody effective, when it reacts immunospecifically with prostate or lung cancer cells, to inhibit growth or viability of the cells.

The GPR110 antibody may be a human or humanized anti-GPR110 antibody specific against an epitope contained within SEQ ID NO:1. The antibody may be effective, when bound to GPR110 on the surface of prostate or lung cancer cells, to promote antibody-dependent cell cytotoxicity. The antibody may have conjugated thereto, a therapeutic agent effective to kill or inhibit cancer cells, when the agent becomes bound to or incorporated into said cells.

Further disclosed is a method of reducing tumor burden in a subject with prostate or lung cancer, by the steps of: (a) exposing subject antigen-presenting cells to human GPR110 polypeptide or antigenic fragment(s) thereof, and (b) by this exposing, stimulating and causing clonal expansion of CD4 helper T cells, CD8 Tc cytotoxic lymphocytes and CD8 non-cytotoxic T-suppressor lymphocytes, thereby causing expansion of GPR110 antigen-specific CD4 helper T cells, GPR110 antigen-specific CD8 Tc cytotoxic lymphocytes and GPR110 antigen-specific CD8 non-cytotoxic T-suppressor lymphocytes in the subject.

The exposing step may include exposing subject antigen-presenting cells ex vivo to the human GPR110 polypeptide or antigenic fragment(s) thereof, under conditions effective to activate the cells, and injecting the activated cells into the subject.

Alternatively, the exposing step may include injecting the subject with the human GPR110 polypeptide or fragments thereof, carried in a suitable adjuvant. In a related aspect, the invention includes a method of reducing tumor burden in a subject with prostate or lung cancer, by injecting the subject with human GPR110 polypeptide or antigenic fragment(s) thereof, carried in a suitable adjuvant.

In still another aspect, the invention includes a method for screening for compounds that may be effective in the treatment of prostate or lung cancer. The method includes the steps of adding each of a series of test compounds to a cell that expresses GPR110 protein on its cell surface, where binding of a GPR110 agonist or antagonist to the cell-surface protein is effective to produce a detectable change in cellular state, and with each test compound added, determining whether such detectable change in cellular state has occurred.

In still another aspect, the invention includes a test for the analyte GPR110, or fragments or variants thereof. The agent includes an anti-GPR110 antibody specific against GPR110 or fragment, and an assay tag or label, preferably covalently bound to the antibody, that can be used to detect and/or quantitate the amount of antibody present, when bound to an analyte GPR110.

These and other aspects, objects, advantages, and features of the invention will become apparent to those persons skilled in the art upon reading the details of the invention as more fully described below.

### BRIEF DESCRIPTIONS OF THE DRAWINGS

FIG. 1 shows the genomic organization of the mouse Gpr110 locus, as view by a customized screen print of the UCSC genome web site browser (February 2006 version of the mm8 gene assembly). Top, base position on chromosome 17. The green vertical handle bar below “PicoSL3” represents a retroviral integration into the locus identified from a single tumor (754S-2). A public domain integration site (68SB8_{—}65_H07-1) is indicated below “RTCGD”.

FIGS. 2A-2C show immunohistochemical stains (brown) of human prostate tumor (2A), benign prostate hyperplasia (2B), and normal tissue (2C). No or low expression is generally seen in normal tissue or benign prostate hyperplasia, while significant overexpression-is seen in tumor tissue. The polyclonal rabbit antibody serum reacts with an epitope found within amino acid residues 1-590 of human GPR110, defined herein as SEQ ID NO: 1.

FIGS. 3A and 3B show immunohistochemical stains (brown) of human benign prostate hyperplasia tissue stained with anti-GPR110 antibodies (3A) and anti-PSA antibodies (3B). The arrow points to a small group of cancer stem cells that are GPR110-positive and PSA-negative. The GPR110 peptide serum used is the same as described in FIG. 2.

FIGS. 4A and 4B show immunohistochemical stains (brown) of human lung tumor (4A) and normal tissue (4B). No or low expression is seen in normal tissue while significant overexpression is seen in tumor tissue. The peptide serum used is the same as described in FIG. 2.

FIGS. 5A and 5B show a solid-phase diagnostic device for determining GPR110 levels in a human patient, at initial (5A) and final stages (5B) of the assay.

FIG. 6 shows a portion of a gene chip useful for diagnosing genetic predisposition to prostate or lung cancer, constructed in accordance with the present invention.

FIGS. 7A and 7B show GPR110 RNA expression as measured by quantitative PCR in two different sets of normal and tumor lung tissues. The gene GUSB was measured as an endogenous control. Expression was calculated relative to the average expression in the normal lung samples for each set of samples.

### DETAILED DESCRIPTION OF THE INVENTION

### A. Definitions

The following terms have the definitions given below, unless otherwise indicated in the specification.

“Screening” for cancer means diagnostic information that either alone, or in combination with other diagnostic information, can be used to determine the presence or absence of a cancer, or the increased likelihood of a cancer, or used to classify the type of cancer, e.g., a lung cancer characterized by elevated levels of GPR110 expression.

“Other indicator that is diagnostic of lung or prostate cancer” refers to a diagnostic test, other than a biological marker that can be used to detect or characterize the presence or extent or type of a cancer. Exemplary indicators include imaging data obtained by X-ray, CT scan, or MRI imaging methods, or histological observations on biopsied tissue.

“Staging” treatment of cancer, in accordance with the present invention, involves determining the stage of cancer in an individual, based on the level of GPR110 detected, and tailoring the treatment to that stage. There are four recognized stages of cancer, which are defined by the degree of localization and organization of cancer cells. In addition, cancer may be defined as early stage at which the cancer is responsive to a number of hormonal-based therapies, and a later, more serious androgen-independent stage.

An “assayed level of GPR110” refers to an assayed level of wildtype human GPR110, or a variant, e.g., splice variant or mutated form of the protein, or a GPR110 fragment.

An “assayed level of human GPR110 transcript” refers to an assayed level of RNA transcript encoding wildtype human GPR110, or a variant, e.g., splice variant or mutated form of the protein, or a GPR110 fragment.

An “increased” or “above-normal” level of GPR110 refers to a level of the protein, or fragment or variant thereof, as determined, for example, by immunochemical staining or detection that is at least about 50 percent higher than the value of the detectable level of the protein measured in a population of normal (non-cancerous) individuals. Preferably, the level of GPR110 is at least three times higher than the GPR110 value for a similar sample from a normal patient.

An “increased” or “above-normal” level of GPR110 RNA transcript refers to a level amount of the transcript, as determined, for example, by PCR amplification and transcript separation that is at least about 50 percent higher than the value of the detectable level of the transcript measured in a population of normal (non-cancerous) individuals. Preferably, the level of GPR110 is at least three times higher than the GPR110 value for a similar sample from a normal patient.

“The value of the detectable level of GPR110 protein or its RNA transcript measured in a population of normal (non-cancerous) individuals” may refer, for example, to the statistical mean or average of such values for a population, e.g., 5 or more, preferably 10 or more normal individuals, or may refer to the highest value recorded for the GPR110 protein or transcript for the individuals in the population of normal individuals. Such values are readily determined by assaying GPR110 or its transcript from a selected sample source, e.g., lung or prostate tissue, or a blood or serum sample, using assay methods described below. It will be understood that normal values are determined from the same type of tissue, e.g., lung or prostate tissue, or sample source, e.g., a blood or serum sample, as the tissue or sample source being assayed for the presence of elevated levels of GPR110 or its transcript.

“GPR110 assay” refers to an assay that measures the level or presence of GPR110 protein, either in wildtype or variant form, or an epitope thereof, or measures the level of an RNA transcript that encodes GPR110 protein or a fragment thereof.

### B. GPR110 Protein and Expression

The human GPR110 gene encodes a putative orphan “adhesion class” G protein-coupled receptor whose biological function and natural ligand(s) are unknown (refs. 1-3). The human GPR110 gene has two known isoforms and is found at chromosome region 6p12.3. Isoform 1 (NM_{—}153840.2) encodes a putative protein (NP_{—}722582.2) having 910 amino acids (AA) and a calculated molecular weight (MW) of 101234 Da. Isoform 2 (NM_{—}025048.2) encodes a putative protein (NP_{—}079324.2) having 218 AA, a calculated MW of 24745 Da, and a unique C-terminus compared to isoform 1. The mouse Gpr110 gene (NM_{—}133776.1) is found at chromosome region 17 B3; the encoded protein (NP_{—}598537.1) has 908 AA and a calculated MW of 101338 Da. The human GPR110 protein is a putative cell surface 7 transmembrane protein, and contains a G-protein-coupled receptor proteolytic site (GPS) domain and an SEA domain, as well as several possible N-linked glycosylation sites near the N-terminus.

### C. Identification of GPR110 as a Cancer Gene

Cancer genes (oncogenes and tumor suppressor genes) were defined in a high throughput manner by using proviral tagging. Although viruses have not yet been implicated as a major cause of cancers in humans, research using tumor viruses has led to the discovery of many oncogenes and protooncogenes. In proviral tagging, mice are infected with a retrovirus that does not contain an oncogene (e.g., murine leukemia virus, MLV or murine mammary tumor virus, MMTV (4-8). Recently, the host range of this approach has been broadened by the use of a transposon (9, 10).

During retroviral infection, the virus integrates into the cellular genome and inserts its DNA near or within genes, which leads to various outcomes: (i) the insertion site is too far away from a protooncogene and thus does not activate it. In this case, there will be no selection for that cell. (ii) The provirus inserts within 200 kb of a protooncogene, but not within the gene (type 1). Here, either the viral promoter or the viral enhancer increases the expression level of the protooncogene. (iii) The provirus inserts within a gene, destroying or altering its function (type 2). There will be no selection for a cell that contains either type 1 or type 2 insertion events in a gene that is not a protooncogene or tumor suppressor gene. If integration results in the formation of a tumor, genes adjacent to the integration site can be identified, and classified as either protooncogenes or tumor suppressor genes. This method has been used to identify many new protooncogenes as well as to confirm already known protooncogenes discovered by virtue of their homology to viral oncogenes (7, 8). A tumor suppressor may be scored if a retrovirus lands within a gene and truncates or destroys it. In these cases, the suppressor may be haplo-insufficient, or alternatively, the mutation on the other allele is provided spontaneously by the mouse. The integration event may also lead to more complex consequences, such as a dominant negative effect of the truncated gene product or the transcription of anti-sense or microRNA.

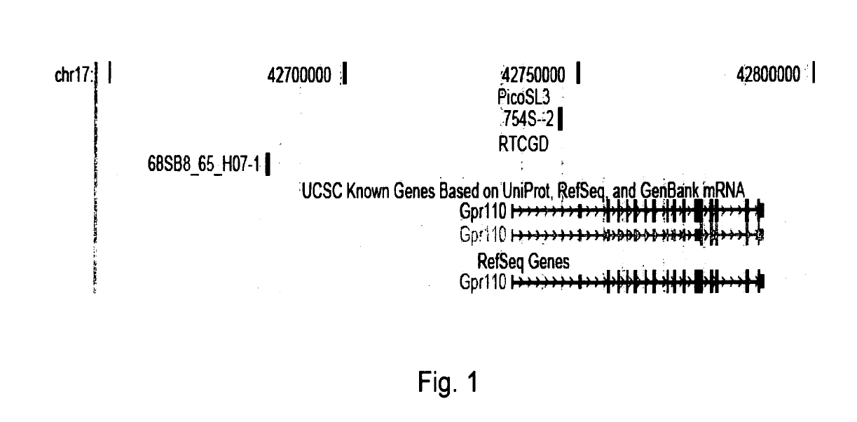

In a screen with T lymphotropic virus SL3-3, a mouse tumor was recovered that contained a proviral integration within intron 1 of the Gpr110 gene (FIG. 1). This integration causes the overexpression of the Gpr110 gene. The human ortholog of this gene is the human GPR110 gene.

### D. Expression of GPR110 and RNA Transcript in Human Tumors and in Normal Tissue

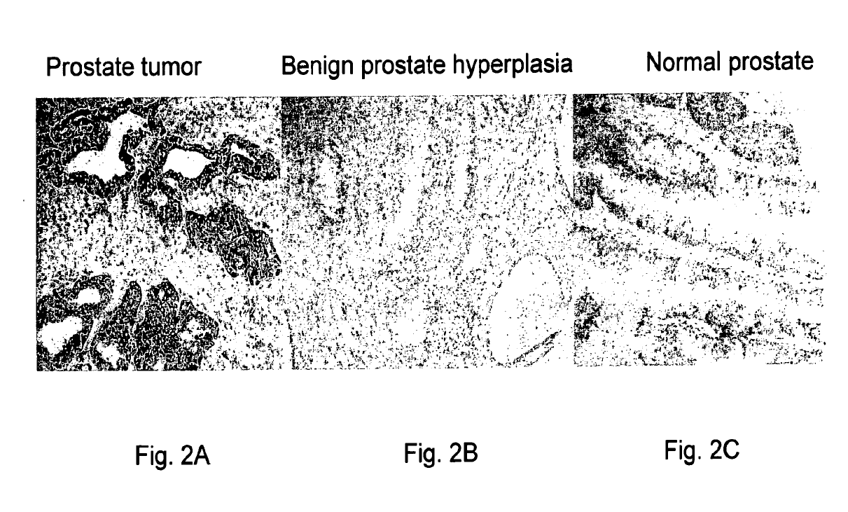

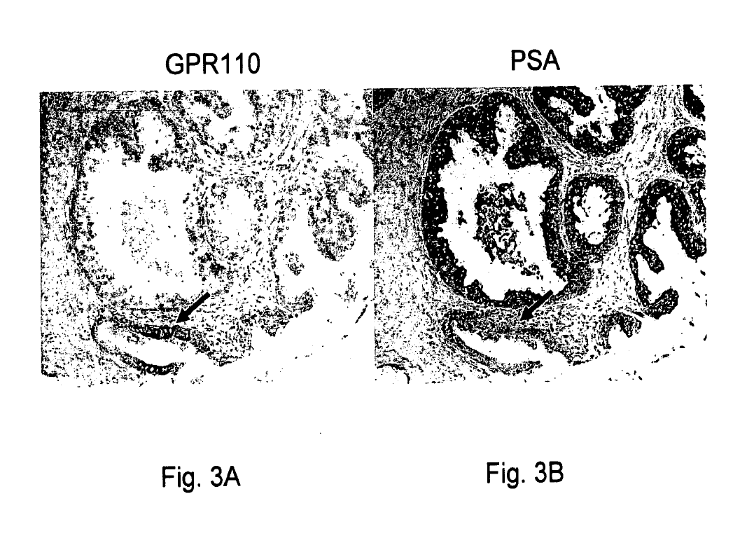

The antigenic epitope to the GPR110 antibody is over-expressed in tumors of the human prostate (FIG. 2A), whereas benign prostate hyperplasia (BPH) cells and the normal counterpart of the prostate tumor cells do not or only weakly express the GPR110 protein (FIG. 2B, 2C), demonstrating that an increased distribution and/or localized amount (density) of the protein is diagnostic of human prostate cancer. On occasion, a small subset of BPH cells stain with the GPR110 antibody (FIG. 3A, arrow). These GPR110-positive cells lack PSA expression (FIG. 3B, arrow) and are consistent with being classified as prostate cancer stem cells.

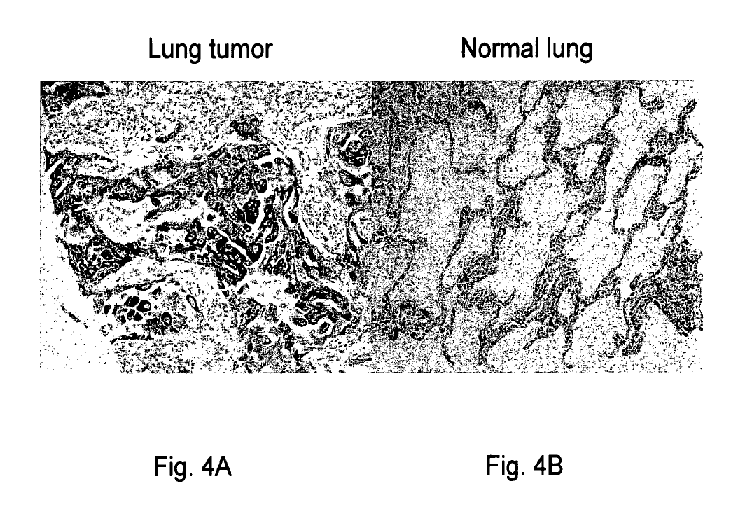

Furthermore, the antigenic epitope to the GPR110 antibody is also over-expressed in human tumors of the lung (FIG. 4A), whereas the normal counterpart of these tumor cells do not or only weakly express the GPR110 protein (FIG. 4B), demonstrating that an increased distribution and/or localized amount (density) of the protein is diagnostic of human lung cancer.

More generally, the invention provides a method for examining tissue or other subject sample, e.g., blood or serum, that normally only weakly expresses or contains GPR110, for the presence and extent of cancer. The method is especially useful for examining prostate and lung tissue, e.g., for determining a subtype of prostate and lung cancer in a human patient. In one method directed to examining prostate and lung tissue, the tissue is stained with a labeled antibody specific against a selected domain or epitope of GPR110, e.g., fluorescence-labeled antibody (see Section E below), to attach the marker to the tissue cells. Alternatively, the tissue is stained with an unlabeled GPR110 antibody, and the cell-bound antibody complex is labeled with a second labeled antibody, e.g., a second antibody carrying a fluorescent, colorimetric or gold-particle reporter. The presence, extent, and stage of prostate or lung cancer in the tissue is then determined based on an increased distribution and/or extent, and typically both, of detectable marker with respect to the distribution and extent of marker in normal prostate or lung cells. Scoring methods for scoring the degree and extent of antibody binding to a histological tissue sample are well known (e.g., “Loda System”). In the present method, intensity scores of 2+ or 3+ and a % cell staining score of 2 or 3 were observed in 35-40% of lung tumors labeled with an anti-GPR110 antibody. For prostate tumors, 20% of tumors had an intensity score of 1 and % cell staining score of 2. For benign prostate hyperplasia samples, 70% had both an intensity score and % cell staining score of 0; 30% had an intensity score of “trace” or 1, with a % cell staining score of 1, when labeled with an anti-GPR110 antibody.

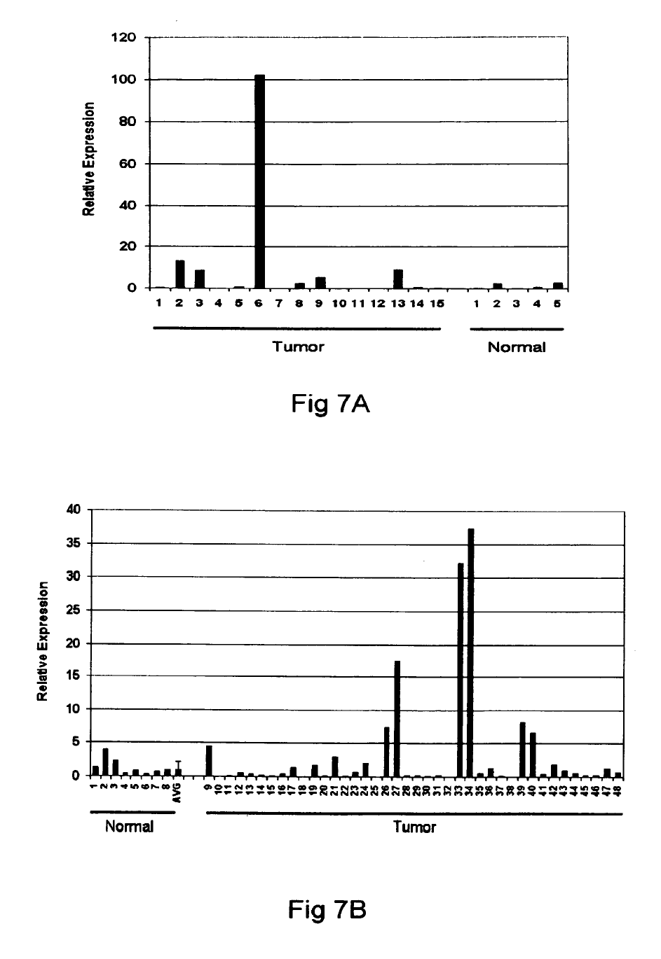

To corroborate GPR110's role in lung cancer, GPR110 RNA transcript levels were measured (using an exon junction (ExJ2-3) Taqman probe) in two different sets of normal and tumor lung tissues. In the first set of tissues, four of the 15 lung adenocarcinoma tumors assayed (2, 3, 6, and 13) showed 8-fold to over 100-fold higher GPR110 RNA levels than normal lung samples (FIG. 7A). In the second set of tissues, six of the 40 lung cancer samples had from 5-fold up to 35-fold overexpression of GPR110 (FIG. 7B) as compared to the normal lung samples. Four of these six elevated samples (27, 33, 34, and 39) were from lung adenocarcinomas while the remaining two (26 and 40) were squamous cell carcinomas. Overall, from both expression experiments, elevated GPR110 expression was seen in ˜20% of lung tumors assayed.

### E. Preparation of Anti-GPR110 Antibody

This section describes production of anti-GPR110 antibodies useful for diagnostic and therapeutic purposes, as described further in the sections below. The anti-GPR110 antibody used in the present invention can be obtained by any variety of conventional methods to produce a monoclonal, polyclonal, and/or recombinant antibody. One preferred antibody, particularly for diagnostic use, is a mouse monoclonal antibody, prepared according to well-known hybridoma methodology. Briefly, human GPR110 may be first obtained, for example, by expressing the GPR110 gene. The purified GPR110 protein acts as an immunogen. Alternatively, a partial peptide of GPR110 can be used as a sensitization antigen. In particular, for generating antibodies specific against a selected epitope or domain of GPR110, a peptide defining that domain or epitope may be used as the immunogen. Exemplary immunogens include the GPR110 epitope represented by amino acid residues within SEQ ID NO:1.

Anti-GPR110 antibodies useful in diagnostic applications may be labeled with a variety of detectable labels, including detectable reporters, such as enzymes for enzyme-linked immunosorbent assays (ELISA), detectable particles, such as gold particles and reporter-carrying liposomes, colorimetric or fluorescent reporters, labels such as quantum dot nanocrystal particles, radiolabels, and labels such as a biotin label by which secondary detectable labels, such as a reporter-labeled streptavidin label can be attached. In some assay formats, an unlabeled anti-GPR110 antibody, for example, a mouse IgG antibody, is detected by reaction with a labeled antibody, e.g., a labeled anti-mouse IgG antibody.

For therapeutic uses, human monoclonal antibodies having binding activity to GPR110 can be produced by sensitizing in vitro human lymphocytes with GPR110, and causing the sensitized lymphocytes to fuse with the human-derived myeloma cells having a permanent division potential. Alternatively, GPR110 as an antigen can be administered to a transgenic animal having all the repertories of a human antibody gene to obtain anti-GPR110 antibody-producing cells, and then human antibodies for GPR110 may be obtained from the immortalized anti-GPR110 antibody-producing cells.

Also for therapeutic uses, the antibody may be conjugated to (derivatized with) a therapeutic agent, such as a toxin, radiolabeled metal anchored in chelated form, or carrier body, such as liposomes, loaded with an anti-tumor agent, where localization of the antibody carrier on the surface of cells is effective to cause disruption of the cell membrane, e.g., by fusion of the carrier with the cell membrane, and release of the therapeutic agent into the cells.

In still other methods, human or humanized antibodies specific against GPR110 antigen can be prepared by recombinant techniques, such as have been reported (see, for example, U.S. Pat. Nos. 6,090,382 and 6,258,562).

### F. Diagnostic Methods and Reagents

In one aspect, the invention includes a method of screening for prostate or lung cancer in a human subject, by (a) assaying the level of human GPR110 or its RNA transcript in a subject sample, and (b) determining if the assayed level of human GPR110 or its RNA transcript is at least threefold greater than the level of GPR110 or its transcript, respectively, in a normal human subject, as determined from a plurality of normal human samples. If the assayed level is at least threefold greater than normal level, the method may further involve detecting the presence of lung or prostate cancer by means of an independent test for lung or prostate cancer, respectively, in the subject, where the independent test may be carried out prior to, contemporaneous with, or following steps (a) and (b).

For example, where the independent test precedes the GPR110 assay, the independent test may indicate the presence of lung cancer or prostate tumor, and the GPR110 assay is subsequently employed to confirm the presence of the cancer and/or indicate that the cancer is a type characterized by increased level of GPR110 or its transcript. Where the independent test is carried contemporaneously with the GPR110 assay, the method provides an assay result in which two or more cancer markers, including GPR110 or its transcript, are used to detect lung or prostate cancer in a subject. In a third embodiment, the independent test may be carried out subsequent to the GPR110 assay, to verify the diagnosis of lung or prostate cancer, and/or to indicate that the cancer is a type characterized by the presence of elevated level of GPR110 or a variant thereof.

An embodiment of the method in which the subject sample is a lung or prostate histological tissue sample has been described above. In this embodiment, the sample is prepared for histological examination and stained with an anti-GPR110 antibody specific against a GPR110 epitope, under conditions effective to bind the antibody to cells having the GPR110 epitope. The level of antibody associated with the sample can be determined by standard histological methods, e.g., measurement of overall staining or fluorescence, or measurement of radioactivity levels where the anti-GPR110 antibody is labeled with a radioactive marker.

An embodiment of the method in which the subject sample is a blood or serum sample is detailed below. This embodiment involves contacting the sample with an anti-GPR110 antibody specific against a GPR110 epitope, under conditions effective to bind the antibody to the GPR110 epitope, separating antibody bound to the GPR110 epitope from unbound antibody, and detecting the level of the antibody bound to the GPR110 epitope. Solid-strip assay devices having immobilized anti-GPR-Assay for capture of GPR110 in a sample are discussed below. Preferred body-fluid samples are blood, urine, and saliva. Where urine is assayed, the assayed level of GPR110 indicative of prostate or lung cancer is typically in the range of greater than about 1 ng/ml sample fluid.

A third general embodiment in which the subject sample is a lung or prostate tissue, for assay of GPR110 transcript, is detailed above with reference to FIG. 7A and 7B. In this embodiment, the tissue sample is processed to extract RNA transcript therefrom, and the level of RNA transcript encoding for at least a fragment of GPR110 protein is determined by standard methods, such as sequence-specific amplification by PCR, or other methods involving sequence-specific probes, according to well-known methods.

More generally, detection of GPR110 or its transcript as an aid in diagnosis for the presence, extent, or staging of prostate or lung cancer can be used alone or in combination with the detection and screening of additional marker proteins associated with prostate or lung cancer. Biomarkers or marker proteins refer to any detectable biological molecule in which its altered expression, distribution, or a particular form of the biomarker correlates with the presence, extent, or stage of a physiologic condition, such as a disease state. As understood by those skilled in the art, there need not be a strict association between the biomarker and the physiologic condition, but only that a statistically significant association is present between the biomarker and the physiologic condition. Additional biomarkers can be selected from, among others, prostate specific antigen (PSA), including total or free PSA or both, glypican 3 protein (GPC3) and combinations thereof.

Where the additional biomarker is PSA, the levels or distribution of PSA can be determined, as noted above, for total PSA, free PSA, or combination thereof, according to methods in the art. In some cases, the levels of total PSA and fPSA, generally expressed in the art as the ratio of fPSA to total PSA, can be used in combination with detection of GPR110. Testing for PSA can be on the same or different biological specimen used to detect the GPR110. For example, for use in screening male human subjects for prostate cancer, step (a) in the method described above may include reacting the sample with an antibody specific against prostate-specific antigen (PSA), to produce a reaction product related to the level of PSA in the sample, and step (b) may include determining, level of PSA when compared with a normal range of PSA in non-cancerous human samples.

The additional biomarker GPC3 is characterized as a heparin sulfate proteoglycan anchored to the cell membrane via glycosylphosphatidylinositol. The protein has a molecular weight of 65.6 kDa and the polypeptide chain has 580 amino acid residues. The heparin sulfate chain of the proteoglycans interacts with heparin-binding growth factors and thus serves as a co-receptor in cell signaling, although GPC3 might bind also in a different way. In embryonic development, GPC3 modulates BMP and EGF-mediated effects during renal branching morphogenesis. It also controls cellular responses to BMP4 in limb patterning and skeletal development. The levels of GPC3 protein are increased in prostate cancer tissues. Its use as a specific biomarker for prostate and methods of its detection, such as by antibodies specifically binding to GPC3, are described in co-owned U.S. application Ser. No. 11/325,847. A method for detecting GPC3 can employ antibodies that specifically bind to GPC3, such as polyclonal, monoclonal, or recombinant antibodies. An exemplary antibody, particularly for diagnostic use, includes a mouse monoclonal antibody, prepared according to well-known hybridoma methodology. Briefly, human GPC3 may be first obtained, for example, by expressing the GPC3 (MXR7) gene as disclosed by Lage, H. et al (Gene 188 (1997), 151-156). The purified GPC3 protein is used as an immunogen. Alternatively, a partial peptide of GPC3 can be used as a sensitization antigen. The partial peptide can be obtained by chemical synthesis from the amino acid sequence of human GPC3. By way of example and not limitation, exemplary GPC3 sequences that may be employed include, among others, DLFIDKKVLKVAHVEHEET, SEQ ID NO:3 (amino acid residues 365 to 383, encoded by exon 4) and LAYDLDVDDAPGNSQQ, SEQ ID NO:4 (amino acid residues 526 to 541, encoded by exon 8). Other peptides for producing antibodies directed against GPC3 will be apparent to the skilled artisan in view of the known sequence of GPC3.

The assay may be carried out by any of a variety of assay methods used for detecting body-fluid antigens, including ELISA techniques, homogeneous assays, for example, involving fluorescence quenching, and a variety of solid-phase sandwich assays in which the GPR110 antigen is captured by an anti-GPR110 antibody carried on a solid support, and the immobilized antigen-antibody complex is labeled with a second anti-GPR110 antibody, e.g., a second antibody carrying a colorimetric or gold-particle reporter.

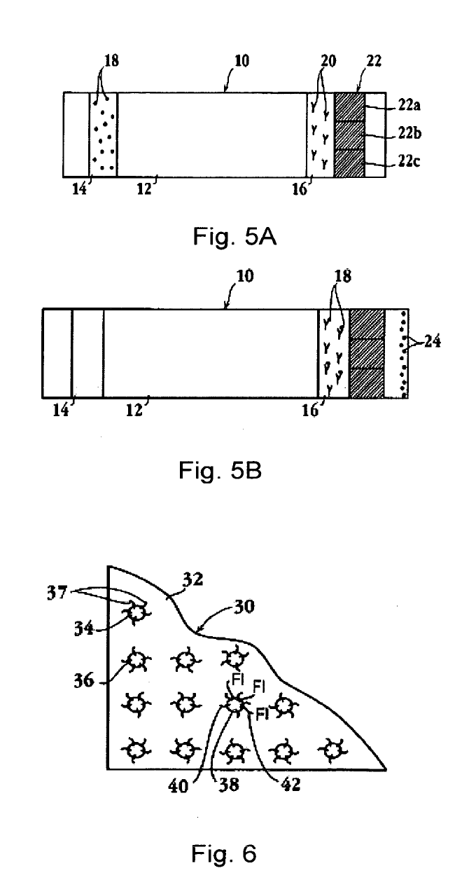

FIGS. 5A and 5B illustrate a solid-phase assay strip constructed in accordance with an embodiment of the invention, suitable for carrying out a sandwich immunoassay of the type just mentioned, and shown in initial and final assay states, respectively. The strip, indicated generally at 10, includes a porous support or pad 12 having a sample-application zone 14 in an upstream region of the support and a sample-detection zone 16 in a downstream region. The sample-application zone includes a detectable anti-GPR110 antibody reagent, e.g., anti-GPR110 antibodies labeled with gold particles, and carried in the zone in an unbound, i.e., non-immobilized form. This reagent is indicated by solid circles, such as at 18. Anti-GPR110 antibodies, which may be the same or different from those in the labeled antibody reagent, are immobilized to the solid support within the detection zone, and are indicated by the “Y” shapes, such as at 20.

Also shown is a reference zone 22 which is located adjacent the detection zone and has one or more colored or shaded regions corresponding to different assay levels of GPR110 in a body-fluid sample. In the embodiment shown, zone 22 includes three regions 22a, 22b, and 22c, corresponding to an assayed level of GPR110 (a) below that associated with cancer, (b) corresponding to a lower threshold level associated with cancer, and (c) a level that is substantially higher, e.g., 2-3 times, higher than the threshold layer in region 22b, respectively. These three regions provide a known standard indicator against which the level of detectable reaction produced can be assessed as a level associated with prostate or lung cancer. Together, the assay strip and reference zone constitute an assay device for use in screening for prostate or lung cancer in a human subject, or for staging treatment of prostate or lung cancer in a human subject.

In operation, a known volume of a body-fluid sample to be tested is added to the sample-application zone of the strip, where it diffuses into the zone, allowing the antibody reagent to react with GPR110 antigen in the sample to form an antigen-antibody complex. This complex and unbound antibody reagent then migrate downstream by capillarity toward the detection zone, where the antigen-antibody complex is captured by the immobilize antibody and the unbound reagent is carried to the end of the support, as indicated at 24. As can be appreciated, the higher the concentration of antigen in the body fluid, the higher the density of captured reagent in the detection zone and the greater the color or intensity in this zone. This color or intensity produced in the detection zone is compared with the standards in the reference zone to determine a qualitative level of GPR110 associated with the presence or absence of prostate or lung cancer. If an above-threshold level of GPR110 is observed in the assay, the subject can be classified in a higher-probability category for the presence of cancer and the subject may be recommended for additional testing and/or more frequent testing.

In another embodiment, the assay device includes an assay strip like that described above, but where the known-reference indicator is provided by a strip-reader instrument reader having (i) a reader slot for receiving the assay strip, (ii) a light source and an optical detection, e.g., a spectrophotometric detector, for detecting an assay-related optical condition at the detection zone of the assay strip, (iii) an electronics or processor unit which records and processes a signal from the optical detector, and converts the signal to an assayed level of GPR110, and (iv) a user display screen or window. The instrument may report the actual GPR110 body-fluid sample detected, allowing the operator to compare the displayed value with known standard indicator levels provided with the assay strip or instrument, to assess whether the subject has an increased GPR110 level associated with prostate or lung cancer, or to assess the possible stage of the cancer, for purposes of treatment design. Alternatively, the instrument itself may contain stored known-standard indicator levels which can be compared internally with an assayed level to generate an output that indicates whether an increased GPR110 level associated with prostate or lung cancer has been detected, or to indicate the stage of the cancer.

It will be appreciated how the just-described assay device may be modified for detecting multiple marker proteins, and in particular, for screening of or detection for prostate cancer, the GPR110 protein in combination with total PSA, free PSA and/or GPC3. Each marker may be measured in a separate strip to which the marker-specific antibody is confined, and the device may further include multiple reference zones, each providing a known-standard indicator against which the level of detectable marker protein reaction produced can be assessed, in combination with the level of detectable GPR110, as an indicator of prostate cancer.

Alternative, in an electronic assay device, the reference values of the multiple markers may be stored or represented as tuples of values, e.g., pairs of values, where each value in a tuple represents an indicator value for cancer for a given marker, so that by analyzing the multi-values assay results against the stored tuple values, the device can make a determination of cancer risk based on correlations with a greater number of markers.

### G. Identifying Genetic Mutation Associated With Cancer

In another aspect, the invention provides a method for identifying mutations associated with increased risk of cancer, such as prostate or lung cancer, in a human subject. The section below is described in relation to prostate or lung cancer; however, it will be appreciated that the method may be practiced for other cancers involving increased expression of GPR110. In practicing the method, genomic DNA is extracted from human patients having prostate or lung cancer, preferably including patients from men or women representing different racial and age groups. The DNA sequences or regions that are examined, in particular; are (i) the promoter or 5′ UTR region within 15 kB or less of exon 1 of the human GPR110 gene, (ii) a 3′ UTR region within 5 kB or less of exon 15 of the same gene, and (iii) within exons 1-15 of the same gene.

Mutations, including gene amplifications, at one or more sites along the region are identified by comparing each of the sequences with sequences from the same region derived from normal (wildtype) prostate or lung tissue. Preferably sequences from a number of wildtype individuals are determined to ensure a true wildtype sequence. For each extracted DNA, the patient and wildtype sequences are compared to identify mutations in the patient sequences, and thus mutations that are likely associated with increased risk of prostate or lung cancer.

Once a large number of these mutations are identified, e.g., at least 50-200 or more, they may be used in constructing a genetic screening device, e.g., a gene chip, useful for screening individuals for genetic predisposition to prostate or lung cancer. In one embodiment, the device includes a gene chip, such as shown at 30 in FIG. 6, having an array of regions, such as regions 34, 36, each containing bound known-sequence fragments, such as fragment 37 in region 34. The fragments or probes are preferably 25-70 bases in lengths, and each includes one of the above-identified mutations upstream of the GPR110 gene that is associated with prostate or lung cancer. Gene-chip construction and detection of mutant sequences with such chips are well known.

In a typical genetic-screening procedure, patient cells are obtained, genomic DNA is extracted, and sequence regions of interest are amplified by standard PCR, employing fluoresceinated probes. The amplified material is then reacted with the chip-array sequences, under suitable hybridization conditions, and the array surface is washed to remove unbound material, and then scanned with a suitable chip reader to identify any mutated sequences associated with prostate or lung cancer. The figure shows binding of a labeled genomic DNA fragment, indicated at 42, to an array region 38 having bound probe molecules 40. Detection of a fluorescent signal in this array region is diagnostic of a known genetic mutation in the critical upstream GPR110 region may be diagnostic of a genetic predisposition to prostate or lung cancer.

In an alternative embodiment, the mutations identified as above are used to construct a set of molecular inversion probes (MIPs) capable of identifying the presence of genomic mutations. The construction and use of MIPs for identifying genetic mutations have been described (see, for example, reference 11).

### H. Treatment Methods and Pharmaceutical Preparations

The invention also includes methods for treating, e.g., reducing the tumor burden in a human subject, a cancer characterized by an increased expression of GPR110 in the cancer cells. The section below is described in relation to prostate or lung cancer; however, it will be appreciated that the method may be practiced for other cancers characterized by increased expression of GPR110.

In one approach, a GPR110 antigen, e.g., full length GPR110 or an antigenic peptide, such as one of the GPR110 peptides disclosed above, e.g., containing an amino sequence from one of SEQ ID NO: 1, is used to activate immune cells that participate in inducing cytotoxic T cells specific against prostate or lung cancer cells. This may be done, in one embodiment, by exposing antigen-presenting cells obtained from the patient ex vivo with the GPR110 antigen, under conditions effective to activate the cells, e.g., in the presence of GM-CSF. Once activated ex vivo, the cells are reintroduced into the patient, where the activated cells are effective in stimulating clonal expansion of cytotoxic T cells against the tumor. This immunotherapy approach is described, for example, in U.S. Pat. No 6,080,409 and pertinent references cited therein.

Alternatively, the GPR110 antigen may be administered to the patient as a vaccine, typically present in a suitable adjuvant, such as one containing GM-CSF. The peptide vaccine is effective to stimulate and causing clonal expansion of CD4 helper T cells, CD8 Tc cytotoxic lymphocytes and CD8 non-cytotoxic T-suppressor lymphocytes, causing expansion of GPR110 antigen-specific CD4 helper T cells, GPR110 antigen-specific CD8 Tc cytotoxic lymphocytes and GPR110 antigen-specific CD8 non-cytotoxic T-suppressor lymphocytes in the subject.

Preparation of antigen-containing compositions suitable for injection, and suitable antigen doses for immuno-stimulation of cytotoxic T cells have been described in a number of patents and literature publications on T-cell induction by immunotherapy. Those methods are applicable in the present method involving GPR110 antigen for the treatment of prostate or lung cancer. Following treatment, the patient is monitored for change in status of the cancer, typically by a combination of a tumor-visualization procedure, such as MRI or CAT scan, and levels of prostate or lung-cancer-related antigens, including GPR110 itself.

In a second general immunotherapy approach, a patient diagnosed with prostate or lung cancer is first confirmed as having elevated levels of GPR110, according to assay methods described above. If the subject tests positive in this assay, he or she is treated by administration of anti-GPR110 antibody. Preferably the antibody is a human or humanized antibody, prepared as described above, and is administered by IV or subcutaneous injection in a suitable physiological carrier. The antibody dose is preferably 1 to 10 mg/injection, and the patient is treated at intervals of every 14 days or so. During treatment, the patient is monitored for change in status of the cancer, typically by a combination of a tumor-visualization procedure and levels of prostate or lung cancer-related antigens, as above. The treatment may be carried out in combination with other prostate or lung-cancer treatments, including drug or radio-isotope therapy, and may be continued until a desired reduction in tumor size is observed. The GPR110 antibody may be a human or humanized anti-GPR110 antibody, effective, when bound to GPR110 on the surface of prostate or lung cancer cells, to promote antibody-dependent cell cytotoxicity. The antibody may be derivatized with a therapeutic agent, such as a toxin, effective to kill of inhibit cancer cells, when the conjugate is bound to or taken up by the cells.

### I. Cell-Based Compound Screening

Multiple expression systems and assays are typically used to assess G protein-coupled receptor (GPCR) function and identify compounds acting as agonists and antagonists. References 12-14 review current general high-throughput approaches to drug compound screening for GPCRs. In large-scale screening programs, GPCRs have been typically expressed using cell-based recombinant expression systems, including yeast, insect (baculovirus), Xenopus oocytes, and mammalian cell lines. While determination of pharmacological activity of GPCRs was traditionally approached by performing radiolabeled ligand binding assays, simple receptor binding may also be detected using non-radioactive methods such as fluorescence polarization and fluorescence resonance energy transfer.

Functional coupling of GPCRs to downstream signaling pathways may be assessed by standard assays measuring downstream events such as intracellular calcium mobilization. With these cell-based assays, the GPCR of interest is expressed, for example, in mammalian cells along with a promiscuous naturally occurring G protein such as G_{q15/16} (or combinations thereof), or promiscuous engineered chimeric G-proteins, both of which can couple with many GPCRs and transduce signaling events; a rise in intracellular calcium can be measured using standard calcium-sensitive fluorescent dyes. To measure more immediate second messenger signaling molecules such as cAMP and arachidonic acid, gene reporter vectors may be used, where cAMP binding sites, for example, are coupled to luciferase fusion genes. Alternatively, a fluorescent based system can be used to measure the translocation of proteins involved in the desensitization of GPCRs, such as B-arrestin2. Other types of expression systems include GPCR expression in Xenopus melanocytes, where GPCR activity is measured by detecting the dispersion or condensation of endogenous pigment present in the melanocytes.

While the invention has been described with respect to particular embodiments and applications, it will be appreciated that various changes and modification may be made without departing from the invention as claimed.

Sequence Listing

N-terminal extracellular domain of human GPR110 protein
(isoform 1) (residues 1-590)
SEQ ID NO: 1
MKVGVLWLISFFTFTDGHGGFLGKNDGIKTKKELIVNKKKHLGPVEEYQLLLQVT
YRDSKEKRDLRNFLK
LLKPPLLWSHGLIRIIRAKATTDCNSLNGVLQCTCEDSYTWFPPSCLDPQNCYL
HTAGALPSCECHLNNL
SQSVNFCERTKIWGTFKINERFTNDLLNSSSAIYSKYANGIEIQLKKAYERIQGFE
SVQVTQFRNGSIVA
GYEVVGSSSASELLSAIEHVAEKAKTALHKLFPLEDGSFRVFGKAQCNDIVFGF
GSKDDEYTLPCSSGYR
GNITAKCESSGWQVIRETCVLSLLEELNKNFSMIVGNATEAAVSSFVQNLSVIIR
QNPSTTVGNLASVVS
ILSNISSLSLASHFRVSNSTMEDVISIADNILNSASVTNWTVLLREEKYASSRLLET
LENISTLVPPTAL
PLNFSRKFIDWKGIPVNKSQLKRGYSYQIKMCPQNTSIPIRGRVLIGSDQFQRSL
PETIISMASLTLGNI
LPVSKNGNAQVNGPVISTVIONYSINEVFLFFSKIESNLSQPHCVFWDFSHLQW
NDAGCHLVNETQDIVT
CQCTHLTSFSILMSPFVPSTIFPVVKWITY
human GPR110 protein (isoform 1) (residues 1-910)
SEQ ID NO: 2
MKVGVLWLISFFTFTDGHGGFLGKNDGIKTKKELIVNKKKHLGPVEEYQLLLQVT
YRDSKEKRDLRNFLK
LLKPPLLWSHGLIRIIRAKATTDCNSLNGVLQCTCEDSYTWFPPSCLDPQNCYL
HTAGALPSCECHLNNL
SQSVNFCERTKIWGTFKINERFTNDLLNSSSAIYSKYANGIEIQLKKAYERIQGFE
SVQVTQFRNGSIVA
GYEVVGSSSASELLSAIEHVAEKAKTALHKLFPLEDGSFRVFGKAQCNDIVFGF
GSKDDEYTLPCSSGYR
GNITAKCESSGWQVIRETCVLSLLEELNKNFSMIVGNATEAAVSSFVQNLSVIIR
QNPSTTVGNLASVVS
ILSNISSLSLASHFRVSNSTMEDVISIADNILNSASVTNWTVLLREEKYASSRLLET
LENISTLVPPTAL
PLNFSRKFIDWKGIPVNKSQLKRGYSYQIKMCPQNTSIPIRGRVLIGSDQFQRSL
PETIISMASLTLGNI
LPVSKNGNAQVNGPVISTVIQNYSINEVFLFFSKIESNLSQPHCVFWDFSHLQW
NDAGCHLVNETQDIVT
CQCTHLTSFSILMSPFVPSTIFPVVKWITYVGLGISIGSLILCLIIEALFWKQIKKSQ
TSHTRRICMVNI
ALSLLIADVWFIVGATVDTTVNPSGVCTAAVFFTHFFYLSLFFWMLMLGILLAYRII
LVFHHMAQHLMMA
VGFCLGYGCPLIISVITIAVTQPSNTYKRKDVCWLNWSNGSKPLLAFVVPALAIV
AVNFVVVLLVLTKLW
RPTVGERLSRDDKATIIRVGKSLLILTPLLGLTWGFGIGTIVDSQNLAWHVIFALL
NAFQGFFILCFGIL
LDSKLRQLLFNKLSALSSWKQTEKQNSSDLSAKPKFSKPFNPLQNKGHYAFSH
TGDSSDNIMLTQFVSNE
(amino acid residues 365 to 383, encoded by exon 4)
SEQ ID NO: 3
DLFIDKKVLKVAHVEHEET
(amino acid residues 526 to 541, encoded by exon 8)
SEQ ID NO: 4
LAYDLDVDDAPGNSQQ

1. A method of screening for lung or prostate cancer in a human subject, comprising
(a) assaying the level of human GPR110 Or its RNA transcript in a subject sample,
(b) determining if the assayed level of human GPR110 or its RNA transcript is at least threefold greater than the level of GPR110 or its transcript, respectively, in a normal human subject, as determined from a plurality of normal human samples.

2. The method of claim 1, wherein the subject sample is a lung or prostate histological tissue sample, step (a) includes contacting said sample with an anti-GPR110 antibody specific against a GPR110 epitope, under conditions effective, to bind the antibody to cells having the GPR110 epitope, and detecting the level of antibody associated with said sample, and step (b) includes determining if the detected level of antibody associated with the subject lung or prostate tissue sample is at least threefold greater than that of anti-GPR110 antibody associated with human lung or prostate tissue samples, respectively, obtained from normal individuals.

3. The method of claim 2, wherein said antibody is specific against a GPR110 epitope represented by amino acid residues within SEQ ID NO:1.

4. The method of claim 2, wherein the anti-GPR110 antibody in step (a) is a radiolabeled GPR110 antibody, step (a) includes detecting the level of localized radiolabel scintigraphically in said tissue.

5. The method of claim 1, wherein the subject sample is a subject blood or serum sample, step (a) includes contacting said sample with an anti-GPR110 antibody specific against a GPR110 epitope, under conditions effective to bind the antibody to the GPR110 epitope, separating antibody bound to the GPR110 epitope from unbound antibody, and detecting the level of the antibody bound to the GPR110 epitope, and step (b) includes determining if the detected level of antibody bound to the GPR110 epitope is at least threefold greater than that of anti-GPR 110 antibody bound to the GPR.110 epitope present in blood or serum samples obtained from normal individuals.

6. The method of claim 5, wherein step (a) includes applying the blood or serum sample body fluid to a solid-phase immunoassay device, where the level of GPR110 in the sample is indicated qualitatively by a colorimetric or fluorometric indicator, and the determining step includes comparing the indicator with a known standard.

7. The method of claim 1, wherein the subject sample is a lung or prostate tissue sample, step (a) includes processing the sample to extract RNA transcript therefrom and detecting the level of RNA transcript encoding for at least a fragment of GPR110 protein, and step (b) includes determining if the detected level of RNA transcript is at least threefold greater than the detected level of transcript encoding for at least a fragment of GPR110 protein in lung or prostate tissue samples obtained from normal individuals.

8. In a method for screening for the presence of lung or prostate cancer, by detection of a depressed or elevated level of a biological marker or other indicator that is diagnostic of lung or prostate cancer, an improvement comprising
(a) assaying the level of human GPR110 or its transcript in a subject sample, and
(b) determining if the assayed level of human GPR110 or its transcript is at least threefold greater than the level of GPR110 or its transcript, respectively, in a normal human subject, as determined from a plurality of normal human samples, as an additional indicator of the presence of lung or prostate cancer, respectively.

9. The improvement of claim 8, wherein the subject sample is a subject lung or prostate histological tissue, sample, step (a) includes contacting said sample with an anti-GPR110 antibody specific against a GPR110 epitope, under conditions effective to bind the antibody to cells having the GPR110 epitope, and detecting the level of antibody associated with said sample, and step (b) includes determining if the detected level of antibody associated with the subject lung or prostate tissue sample is at least threefold greater than that of anti-GPR110 antibody associated with human lung or prostate tissue samples, respectively, obtained from normal individuals.

10. The improvement of claim 9, wherein said antibody is specific against a GPR110 epitope represented by amino acid residues within SEQ ID NO:1.

11. The improvement of claim 9, wherein the anti-GPR110 antibody in step (a) is a radiolabeled GPR110 antibody, step (a) includes detecting the level of localized radiolabel scintigraphically in said tissue.

12. The improvement of claim 9, wherein the subject sample is a subject blood or serum sample, step (a) includes contacting said sample with an anti- GPR110 antibody specific against a GPR110 epitope, under conditions effective to bind the antibody to the GPR110 epitope, separating antibody bound to the GPR110 epitope from unbound antibody, and detecting the level of the antibody bound to the GPR110 epitope, and step (b) includes determining if the detected level of antibody bound to the GPR110 epitope is at least threefold greater than that of anti-GPR110 antibody bound to the GPR110 epitope present in blood or serum samples obtained from normal individuals.

13. The improvement of claim 12, wherein step (a) includes applying the blood or serum sample body fluid to a solid-phase immunoassay device, where the level of GPR110 in the sample is indicated qualitatively by a colorimetric or fluorometric indicator, and the determining step includes comparing the indicator with a known standard.

14. The improvement of claim 8, wherein the subject sample is a lung or prostate tissue sample, step (a) includes processing the sample to extract RNA transcript therefrom and detecting the level of RNA transcript encoding for at least a fragment of GPR110 protein, and step (b) includes determining if the detected level of RNA transcript is at least threefold greater than the detected level of transcript encoding for at least a fragment of GPR110 protein in lung or prostate tissue samples obtained from normal individuals.

15. The improvement of claim 8, in a method for detecting prostate cancer in a human male subject, by reacting a subject body-fluid sample with an antibody specific against at least one marker protein selected from one of total prostate specific antigen (PSA), free PSA, and glypican 3 protein (GPC3), and determining, as an indicator of prostate cancer, whether the subject has an increased level of at least one of said marker protein.

16. (canceled)

17. A diagnostic device for use in the screening for prostate or lung cancer in a human subject, or staging treatment of prostate or lung cancer in a subject. comprising
(a) structure for receiving a body-fluid sample from the subject,
(b) an antibody specific against a selected domain or epitope of GPR110, and associated with said structure and capable of reacting with body-fluid received in said structure, to produce, in combination with other reagents associated with the structure, a detectable reaction indicative of the presence of GPR110 sample protein containing that epitope or domain, and
(c) a first known-standard indicator against which the level of detectable reaction produced can be assessed as an increased level associated with prostate or lung cancer.

18. The device of claim 17, wherein the structure in the device includes a porous pad having the antibody embedded therein, for reaction with the fluid sample when the sample is applied to the pad, the detectable reaction is indicated by a colorimetric or fluorimetric indicator, and the known standard indicator includes an indicia that represents a level of GPR110 containing the epitope or domain corresponding to that associated with prostate or lung cancer.

19. The device of claim 18, further including a spectrophotometric detector for generating a signal related to the level of GPR110 produced, a microprocessor for comparing the signal with a known-standard signal value associated with prostate or lung cancer, and a display for displaying an output of the microprocessor.

20. The device of claim 18, wherein the anti-GPR110 binding protein in the device is an antibody specific against an epitope contained within SEQ ID NO:1 or SEQ ID NO:2.

21. A method for treating prostate or lung cancer in a subject, comprising
(a) determining whether cancer tissue cells from the subject have an increased level of GPR110 protein or RNA transcript, when compared with a normal range of GPR110 or its transcript in human cells of the same tissue, as an indicator of prostate or lung cancer, and
(b) if the subject has such an increased GPR110 or transcript level, administering a therapeutically effective amount of a GPR110 antibody effective, when it reacts immunospecifically with prostate or lung cancer cells, to inhibit growth or viability of the cells.

22. The method of claim 21, wherein the GPR 110 antibody is a human or humanized anti-GPR110 antibody specific against an epitope contained within SEQ ID NO:1.

23. The method of claim 21, wherein the antibody is effective, when bound to GPR110 on the surface of prostate or lung cancer cells, to promote antibody-dependent cell cytotoxicity.

24. The method of claim 21, wherein the antibody has conjugated thereto, a therapeutic agent effective to kill or inhibit cancer cells, when the agent becomes bound to or incorporated into said cells.
